(19) United States
(12) Patent Application Publication
(10) Pub. No.: US 2019/0357609 A1
(43) Pub. Date: Nov. 28, 2019

(54) AMBIDEXTROUS STRESS-REDUCING GLOVE

(71) Applicant: Ansell Limited, Richmond, Victoria (AU)

(72) Inventors: Jali Lamar Williams, Melaka (MY); Christina Foo Lik Erh, Melaka (MY); David Mark Lucas, Petaling Jaya (MY)

(21) Appl. No.: 16/476,611

(22) PCT Filed: Dec. 20, 2017
(86) PCT No.: PCT/AU2017/000284

§ 371 Date: Jul. 9, 2019

Related U.S. Application Data
(60) Provisional application No. 62/450,877, filed on Jan. 26, 2017.

(51) Int. Cl. A41D 19/00

(52) U.S. Cl. CPC A41D 19/0003; A61B 42/10; A41D 19/0082

(57) ABSTRACT

An ambidextrous stress-reducing polymeric glove, comprising: a thumb having a palmside surface and a backhand surface; a plurality of fingers that includes an index finger, a middle finger, a ring finger, and a little finger, each of the plurality of fingers having a palmside surface and a backhand surface; a palm region; a backhand region; and a stress-reducing ridge on at least one of: the backhand surface of the thumb and the palmside surface of the thumb, the backhand surface of the index finger and the palmside surface of the index finger, the backhand surface of the middle finger and the palmside surface of the middle finger, the backhand surface of the ring finger and the palmside surface of the ring finger, the backhand surface of the little finger and the palmside surface of the little finger, or the palm region and the backhand region.

### FIELD

The disclosure is directed to personal protective equipment and, more specifically, to ambidextrous stress-reducing gloves having stress-reducing ridges and formers for manufacturing ambidextrous stress-reducing gloves having stress-reducing ridges.

### DESCRIPTION OF THE RELATED ART

Polymeric gloves, such as surgical and examination gloves, are made of strong but flexible elastomers, which permit a snug fit to hands. Polymeric gloves are often formed in a shape that approximates the shape of a flattened hand. However, gloves made in this shape are not ergonomic. For example, surgeons wearing surgical gloves may do so for long durations during procedures, which tire the surgeon's fingers and/or hands because the elastic modulus of the elastomer, of which the glove is made, must be overcome to flex or bend the fingers. Moreover, the flexure of fingers may be impeded at the knuckles on both a palmside and a backhand side by a glove. In an attempt to relieve stresses, some wearers use oversized gloves, which unfortunately cause a loss of dexterity and grip, particularly at the fingertips. Furthermore, surgeons and other medical personnel often “double-glove” to provide extra barrier protection, leading to increased fatigue for wearers. Polymer scientists have failed to create elastomers that are sufficiently flexible and sufficiently abrasion and puncture resistant.

Polymeric gloves are formed by dipping hand-shaped formers into elastomeric emulsions, which adhere to the formers. The emulsion coagulates as a coating on the formers and, following a heat curing step, the gloves are stripped from the formers. However, prior to complete coagulation, the emulsion remains flowable on the formers and the emulsion may drip or flow, leading to variations in quality and localized differences in thickness. Specifically, glove formers are dipped finger first into elastomeric emulsions and allowed to drip dry. However, such gloves are negatively impacted by material bunching in certain areas, such as the fingertips causing, for example, a lack of tactility at the fingertips. Inverting the former can cause bunching at the crotches between the fingers and/or index finger/thumb. Other attempts to create gloves that alleviate stresses on wearers have included gloves having bent fingers, which are bent at the knuckles by 30-50 degrees. However, formers having bent fingers exacerbate differences in thickness of the glove, particularly at the bent areas, leading to stiffer gloves. The formers having bent fingers, in addition to exacerbating differences in thickness, may also trap air, resulting in unfavorable bubbles in the polymeric gloves, and/or trap polymeric material after the gloves are stripped from a former, i.e., quality defects.

Therefore, gloves that can alleviate stresses on the hands of wearers without loss of barrier properties, tactility, and dexterity represent an advance in the art.

### SUMMARY

Ambidextrous stress-reducing gloves, and methods for manufacturing ambidextrous stress-reducing gloves, according to embodiments of the disclosure, substantially as shown in and/or described in connection with at least one of the figures, are disclosed. Various advantages, aspects, and novel features of the present disclosure will be more fully understood from the following description and drawings. The foregoing summary is not intended, and should not be contemplated, to describe each embodiment or every implementation of the present disclosure. Other and further embodiments of the present disclosure are described below. Furthermore, changes and modifications can be made to embodiments described herein without departing from the spirit and scope of the present disclosure and without demising the attendant advantages.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments. It is to be understood that elements and features of one embodiment may be in other embodiments without further recitation. It is further understood that, where possible, identical reference numerals have been used to indicate comparable elements that are common to the figures.

FIG. 1 depicts a perspective view of an ambidextrous stress-reducing polymeric glove having stress-reducing ridges, according to embodiments of the disclosure;

FIG. 2 depicts a side view of stress-reducing ridges disposed on an ambidextrous stress-reducing polymeric glove, according to embodiments of the disclosure;

FIG. 3 depicts a front plan view of a glove former for manufacturing an ambidextrous stress-reducing polymeric glove having stress-reducing ridges disposed thereon, according to embodiments of the disclosure;

FIG. 4 depicts a flow diagram of a method for manufacturing an ambidextrous stress-reducing glove having stress-reducing ridges, according to embodiments of the disclosure;

FIG. 5 depicts a perspective view of stress-reducing ridges disposed on a second ambidextrous stress-reducing glove, according to embodiments of the disclosure;

FIG. 6 depicts a front plan view of a second glove former for manufacturing an ambidextrous stress-reducing polymeric glove, according to embodiments of the disclosure; and

FIG. 7 depicts an apparatus for forming an ambidextrous stress-reducing polymeric glove having stress-reducing ridges, according to embodiments of the disclosure.

### DETAILED DESCRIPTION

Embodiments of the disclosure comprise ambidextrous stress-reducing polymeric gloves, such as examination gloves, surgical gloves, and gloves for industrial and household use. Some embodiments of the disclosure comprise stress-reducing ridges for stress reduction in specific areas, such as areas corresponding to joints of the human hand, such as those between, for example, a distal phalange bone and a middle phalange bone, and/or between a middle phalange bone and a proximal phalange bone, and/or at a metacarpophalangeal joint (MCP), and/or the like. Ambidextrous stress-reducing gloves can be made to reduce stresses on the human hand during flexing and prolonged periods of use while remaining tight and snug at, e.g., fingertips, MCPs, and/or wrists. Furthermore, embodiments of the ambidextrous stress-reducing gloves comprising the stress-reducing ridges permit the use of elastomers having a high elastic modulus. Specifically, because the stress-reducing ridges allow the reduction of stress to the hands of a wearer during use, stronger elastomers can be used for enhanced barrier properties, i.e., enhanced chemical resistance, abrasion resistance, and/or puncture resistance. Thicker polymeric gloves, which are also within the scope of embodiments of the ambidextrous stress-reducing polymeric gloves according to the disclosure, are disclosed. Thinner polymeric gloves may comprise a thickness of approximately 0.005 mm to 0.010 mm. Thicker polymeric gloves may comprise any reasonable thickness for polymeric gloves, for example, approximately 0.010 mm to approximately 0.07 mm.

In some embodiments, consistent with embodiments according to the present disclosure, an ambidextrous stress-reducing polymeric glove includes a thumb having a front surface and a back surface; a plurality of fingers, each of the plurality of fingers having a front surface and a back surface; and a backhand region having a back surface, wherein at least one stress-reducing ridge is formed on at least one of the back surfaces of the thumb, the backhand region, or at least one of the back surfaces of at least one of the plurality of fingers. Embodiments of the disclosure further comprise ambidextrous stress-reducing polymeric gloves having stress-reducing ridge(s) on a palmside surface and stress-reducing ridge(s) on a backhand surface corresponding with the palmside surface. Embodiments of the disclosure comprise ambidextrous stress-reducing polymeric gloves having stress-reducing ridge(s) on a palmside region and stress-reducing ridge(s) on a backhand region corresponding with the palmside region. Some embodiments of the disclosure comprise a bulbous contiguous stress-reducing former ridge that traverse at least 30%, 40%, or 50% of a circumference of a surface of the thumb.

Before describing embodiments of the present disclosure in detail, the terminology used herein describes exemplary embodiments only, and is not intended to be limiting. The disclosure should not necessarily be limited to specific compositions, materials, designs or equipment, as such may vary within the scope of embodiments of the disclosure. All technical and scientific terms used herein have the usual meaning conventionally understood by persons skilled in the art to which this disclosure pertains, unless context defines otherwise. Also, as used in this specification and the appended claims, the singular forms “a,” “an,” and “the” include plural referents unless the context clearly dictates otherwise.

The term “flexing” or “flex” refers to finger movements, such as bending fingers, making a fist, gripping, grasping, clenching or otherwise folding the fingers.

The terms “latex,” “emulsion,” “dispersion,” and “suspension” are generally analogous and indicate a system in which small particles of a substance, such as rubber particles, are mixed with a fluid (such as water and/or alcohols and/or other organic fluids) but are at least partially undissolved and kept dispersed by agitation (mechanical suspension) and/or by the molecular forces (colloidal suspension). Emulsions contemplated herein may further comprise typical and suitable components for rubber or elastomeric formulations, compositions, and/or compounds Suitable components may include accelerators, such as guanidines, thiazoles, thiurams, sulfenamids, thioureas, dithiocarbamates, and xanthanates. Suitable components may include surfactants, such as sodium dodecyl sulfates and polyvinyl alcohols. Suitable components may include activators, such as zinc oxides, cross-linking agents and curatives, such as elemental sulfur and/or polysulphidic donors. Suitable components may include anti-oxidants, anti-ozonants, rheology-modifiers, such as various clays and aluminosilicates. Suitable components may include pH adjusters, such as hydroxides, such as potassium hydroxide, pigments, processing agents, and/or fillers as are known to those in the art.

The term “polymer” generally includes, but is not limited to, homopolymers, copolymers, such as for example, block, graft, random and alternating copolymers, terpolymers, etc. and blends and modifications thereof. Furthermore, unless otherwise specifically limited, the term “polymer” includes all possible geometrical configurations of the molecule. These configurations include, but are not limited to isotactic, syndiotactic and random symmetries.

The term “thermoplastic” generally includes polymer materials that become reversibly pliable, moldable, and/or heatable above a specific temperature and solidify upon cooling. The term “thermoset” generally includes polymer materials that strengthen following heating and solidification, but cannot be successfully remolded or otherwise processed after an initial heat-forming. The term “thermoplastic elastomer” (TPE) connotes a class of copolymers comprising both thermoplastic and elastomeric/thermoset material properties and generally have crosslinking between adjacent polymeric molecular chains. The term “rubber” generally indicates elastomers produced from natural rubber latexes or synthetic elastomers.

Exemplary thermoplastics include, without limitation, polychloroprenes, butyl rubbers, natural rubber, synthetic polyisoprenes, poly(vinyl) chlorides, polyesters, polyamides, polyfluorocarbons, polyolefins, polybutadienes, polyurethanes, polystyrenes, poly(vinyl) alcohols, and copolymers of the foregoing, and elastomeric polymers such as elastic polyolefins, copolyether esters, polyamide polyether block copolymers, block copolymers having the general formula A-B-A′ or A-B like nitrile-butadiene rubber (NBR), styrene-poly(ethylene-propylene)-styrene, styrene-poly(ethylene-butylene)-styrene, (polystyrene/poly(ethylene-butylene)/polystyrene, poly(styrene/ethylene-butylene/styrene), copoly(styrene/ethylene-butylene), A-B-A-B tetrablock copolymers and the like and blends of any of the foregoing.

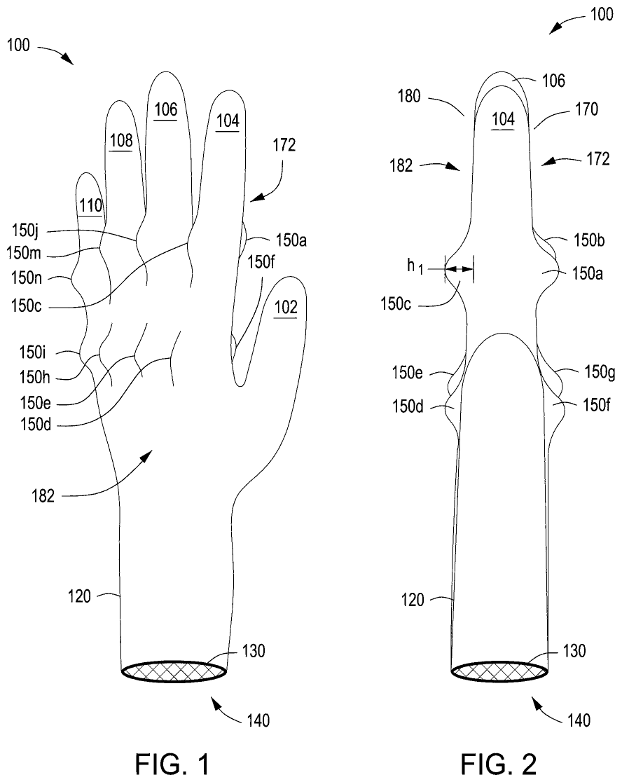

FIG. 1 depicts a perspective view of an ambidextrous stress-reducing polymeric glove 100 having stress-reducing ridges 150, according to embodiments of the disclosure. The ambidextrous stress-reducing polymeric glove 100 comprises a thumb 102, a plurality of fingers, including an index finger 104, a middle finger 106, a ring finger 108, a little finger 110, a backhand region 182 opposite a palmside region 172, and a cuff 120. The ambidextrous stress-reducing polymeric glove 100 optionally comprises a bead 130. The bead is proximate an opening 140 of the ambidextrous stress-reducing glove 100 that defines a volume for receiving a hand of a wearer. Any of the thumb 102, any of the plurality of fingers 104, 106, 108, 110, the palmside region 172, and/or the backhand region 182, may comprise a ridge 150, i.e., 150a-150n, as depicted in FIG. 1. The cuff 120 may comprise one or more ridges 150.

As shown, the ambidextrous stress-reducing polymeric glove 100 comprises at least one stress-reducing ridge 150 on the plurality of fingers 104, 106, 108, 110, the palm region 172, and the backhand region 182. Specifically, the index finger 104 comprises ridge 150a on the palmside region 172. FIG. 1 also shows ridge 150c on the backhand side 182 of the index finger 104. Furthermore, FIG. 1 shows ridge 150f on the palmside region 172 below ridge 150a. Similarly, FIG. 1 depicts a ridge 150d below the ridge 150c on the backhand region 182. A ridge 150e is below a ridge 150j on the middle finger 106, corresponding to the MCP joint. A ridge 150h is below a ridge 150m on the ring finger 108, corresponding to the MCP joint. A ridge 150i is below a ridge 150n on the little finger 110, corresponding to the MCP joint.

FIG. 2 depicts a side view of stress-reducing ridges 150 disposed on the ambidextrous stress-reducing polymeric glove 100, according to embodiments of the disclosure. The side view of the ambidextrous stress-reducing polymeric glove 100 comprises at least one stress-reducing ridge 150 on the plurality of fingers 104, 106, 108, 110, the palm region 172, and the backhand region 182. Specifically, as shown, the index finger 104 comprises ridge 150a and the middle finger 106 comprises ridge 150b of the palmside region 172. FIG. 2 also shows ridge 150c on the backhand side 182 of the index finger 104. Furthermore, FIG. 2 shows ridge 150f on the palmside region 172 below ridge 150a, and ridge 150g on the palmside region 172 below ridge 150b, which corresponds with where the metacarpal bones of the hand meet the carpal bones of the hand, i.e., the MCP joint. Similarly, FIG. 1 depicts the ridge 150d below the ridge 150c on the backhand region 182. The ridge 150e is below the ridge (not shown) on the middle finger 106, corresponding to the MCP joint. Although reference is made to the palmside region 172 and the backhand region 182, the ambidextrous stress-reducing glove 100 may be worn on either hand, i.e., is ambidextrous. In some embodiments, the ambidextrous stress-reducing glove 100 is essentially symmetrical about a longitudinal axis. In other words, either the palm region 172 or the backhand region 182 of the ambidextrous stress-reducing glove 100 may be worn in either orientation on the opposite hand, i.e., on a right or a left hand. For a left handed glove, the palm region 172 would be disposed on a palm of a wearer or, alternatively, the same ambidextrous stress-reducing polymeric glove 100 could be used on the right hand of a wearer, though the palm region 172 would become the backhand region 182. It is to be understood that although the ambidextrous stress-reducing glove 100 may be worn on either hand, the glove 100 need not be symmetrical.

In some embodiments according to the disclosure, the ridge 150 comprises two peaks and a valley disposed between the two peaks (not shown). Exemplary embodiments according to the disclosure, include wherein the ridge 150 comprises three adjacent peaks and a valley disposed between any two neighboring peaks (not shown), wherein a base of the valley is higher than a base point of a surface of the ambidextrous stress-reducing glove. Furthermore, the ridges 150 may be disposed on one or more regions of the ambidextrous stress-reducing glove 100 corresponding with a distal interphalangeal joint, a proximal interphalangeal joint and/or a metacarpophalangeal joint of a hand of a wearer of the ambidextrous stress-reducing polymeric glove 100.

The ridges 150, as with other ridges described herein, provide additional glove material, i.e., additional surface area, at the joints so that a person wearing the ambidextrous stress-reducing polymeric glove 100 may open and close the hand, i.e., flex, without significantly stretching the material in areas of the ambidextrous stress-reducing glove 100 corresponding to the joints. The ambidextrous stress-reducing glove may be made from any of the thermoplastics or thermoplastic elastomers described above. Exemplary elastomers and compositions for making the ambidextrous stress-reducing polymeric glove 100 having stress-reducing ridges 150 are described below in Table 1.

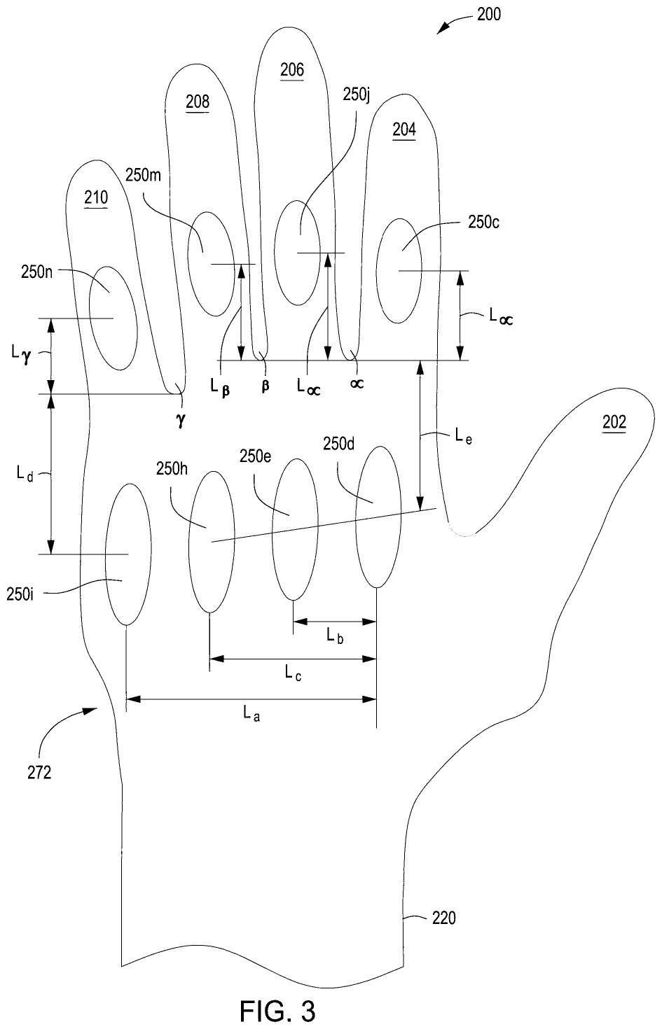

FIG. 3 depicts a front plan view of a glove former 200 for manufacturing an ambidextrous stress-reducing polymeric glove having stress-reducing ridges disposed thereon, according to embodiments of the disclosure. The former 200 comprises a thumb region 202, a plurality of finger regions 204, 206, 208, 210, a palm region 272, a backhand region (not shown), and a cuff region 220. Any of the thumb 202, any of the plurality of fingers 204, 206, 208, 210, the palm region 272, the backhand region, and the cuff 220 may comprise one or more former ridges 250, i.e., 250c-250n for forming stress-reducing ridges on a glove. The glove former 200 is a negative of a glove produced therewith.

As depicted, the glove former 200 comprises a former ridge 250c on the index finger 204, a former ridge 250j on the middle finger 206, a former ridge 250m on the ring finger 208, a former ridge 250n on the little finger 210, a former ridge 250i below the little finger 210, a former ridge 250h below the ring finger 208, a former ridge 250e below the middle finger 206, and a former ridge 250d below the index finger 204. The former 200 also comprises a first crotch α, a second crotch β, and a third crotch γ. The ridge 250c has a peak, approximately at the midpoint of the former ridge 250c, that is located a length La from an axis formed by a line through the first crotch α and the second crotch β. The former ridge 250j has a peak, approximately at the midpoint of the former ridge 250j, that is located a length Lα from an axis formed by a line through the first crotch α and the second crotch β. The former ridge 250m has a peak, approximately at the midpoint of the former ridge 250m, that is located a length L13 from an axis formed by a line through the first crotch α and the second crotch β. The former ridge 250n has a peak, approximately at the midpoint of the former ridge 250n, that is located a length Lγ from the third crotch γ. The former ridge 250i has a peak, approximately at the midpoint of the former ridge 250i, that is located a length Ld below the third crotch γ. The former ridge 250h has a peak, approximately at the midpoint of the former ridge 250h, that is located a length Le from an axis formed by a line through the first crotch α and the second crotch β. The former ridge 250e has a peak, approximately at the midpoint of the former ridge 250e, that is located a length Le from an axis formed by a line through the first crotch α and the second crotch β. The former ridge 250d has a peak, approximately at the midpoint of the former ridge 250d, that is located a length Le from an axis formed by a line through the first crotch α and the second crotch β. The former ridge 250d is approximately a length Lb from a midpoint of the adjacent former ridge 250e, a length Lc from a midpoint of the former ridge 250h, and a length Lα from a midpoint of the former ridge 250i.

In some embodiments of the glove former 200, La may be 50-80 mm. Lb may be 30-50 mm. Lc may be 15-30 mm. Ld may be 12-25 mm. La may be 10-25 min. Lβ may be 10-25 mm. Lγ may be 5-15 mm. It is to be understood that the dimensions are for a size 7.5 former for an ambidextrous stress-reducing glove. The former ridges 150 may be made larger or smaller. Furthermore, a glove itself made from a former may be larger or smaller and the dimensions (size of former ridges 250 and/or lengths between La, Lb, Lc, Ld, Le . . . Lα, Lβ, Lγ, etc.) may be larger or smaller. The peak height h₁ for any former ridge 250 may be 1-20 mm.

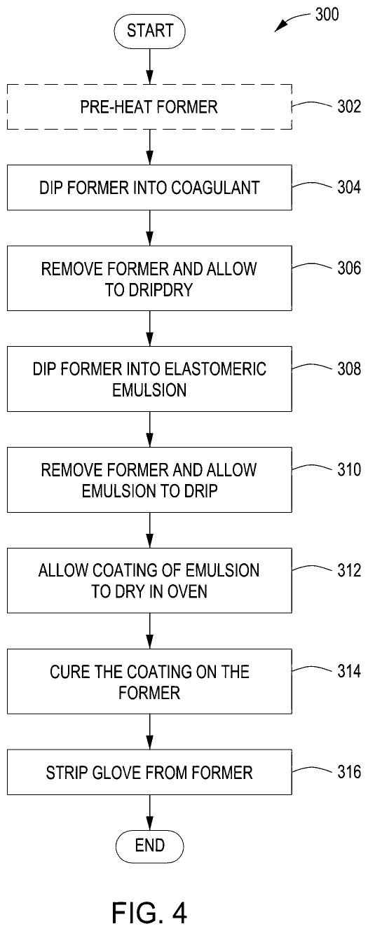

FIG. 4 depicts a flow diagram for a method 300 for manufacturing an ambidextrous stress-reducing polymeric glove having stress-reducing ridges, according to embodiments of the disclosure. At step 302, a hand shaped glove former is provided, which is optionally pre-heated. The glove former is described more fully below and comprises features for making stress-reducing ridges on an ambidextrous stress-reducing glove, as discussed herein, i.e., the ridges have a radius of curvature that are configured to allow excess coagulant solution or emulsion to drip off in a manner that avoids uneven thickness of both the coagulant and/or emulsion, wherein ambidextrous stress-reducing polymeric gloves formed therewith have consistent wall thicknesses.

At step 304, a coagulant is disposed on the glove former, such as the coagulant solution described herein, by dipping the former into a bath of the coagulant solution. In at least one embodiment of the disclosure, the coagulant solution is optionally heated at a temperature ranging from 35-50° C. and, in some exemplary embodiments, from 42-45° C. At step 306, excess coagulant solution is allowed to drip dry from the former and, optionally, the former is vertically rotated so that the fingers of the glove former are pointed up and allowed to drip dry.

At step 308, the glove former has a polymeric or elastomeric emulsion or composition disposed thereon, such as by a dipping step, wherein the glove former is dipped into the emulsion tank in a fingers-first manner. For example, any of the polymeric or elastomeric emulsions or compositions described in Table 1 may be used to manufacture any or all of the ambidextrous stress-reducing gloves described herein. It is to be understood that the amounts, which are expressed in parts per hundred dry weight rubber (PHR), of the components described therein are illustrative of exemplary compositions. Other exemplary embodiments, as are known to those in the art, are within the scope of embodiments described herein. For example, whether expressed as a specific amount or as a range of an amount in Table 1, the values listed can be varied by 20% and produce usable elastomeric compositions. Also, Table 1 lists compositions for natural rubber, synthetic polyisoprene, and polychloroprene. It is to be understood that blends of natural rubber, synthetic polyisoprene, and polychloroprene are within the scope of embodiments of the disclosure.

Additionally, some classes of components are optional, for example, pigments are optional and may be excluded entirely. Also, anti-ozonants are optional. The temperature of the elastomeric emulsion during dipping generally ranges from 15-45° C. and, in at least some exemplary embodiments, ranges from 25-38° C. At step 310, the former is removed from the emulsion tank and the emulsion is allowed to drip down, i.e., fingers are pointing down, to allow excess emulsion to drip off, and reducing the thickness of the elastomeric coating formed by the disposition of the emulsion onto the former and, as above, the former having the elastomeric coating disposed thereon is optionally rotated about a vertical or horizontal axis so that the coating can drip. At step 312, the coating optionally dries in ambient air for several minutes and/or within a heated oven. In at least one embodiment according to the disclosure, the coating is allowed to dry in an oven for several minutes. The drying oven may range from a temperature of approximately 50-70° C.

At step 314, the coating undergoes a curing step to cure the coating, such as within an oven at a temperature of approximately 110-130° C. for ten to twenty minutes. In at least one embodiment according to the disclosure, the coating is cured in a staged process, for example, at a first stage at 90° C. for fifteen minutes, a second stage at 100° C. for fifteen minutes, and a third stage at 130° C. for fifteen minutes. At step 316, a cured glove having stress-reducing ridges is stripped from the former and the method 300 ends. The cured glove may be inverted during stripping or, alternatively, stripped without inverting. Optionally, before the curing step, the elastomeric coating at a distal end from the fingers, i.e., at an open end for receiving a hand, is rolled, e.g., forming a bead, as is known to those in the art.

Some steps of the preceding method 300 may be omitted or performed in a different sequence. Texturization may be imparted using formers that comprise textures at, for example, the fingertips. Moreover, additional steps may be employed, such as a subsequent washing step after curing to remove impurities. Optionally, a coating may be foamed and disposed on the former, as above, and is optionally subjected to a salt texturization process. Also, a second dipping step can provide a second layer of a polymeric coating on any first polymeric coating, wherein either of the first polymeric coating or the second polymeric coating may be foamed. A second polymeric coating may be textured using a salt-texturization process. For example, salt particles may be imbedded into the second polymeric coating of emulsion before the curing step. Thereafter, removing the salt with a solvent, such as water, either before or after a curing step, provides multi-faceted indentations in the coating, which provide enhanced gripping properties, i.e., salt texturization. The technology of providing enhanced gripping properties via salt embedding and removal is disclosed in commonly-assigned U.S. Pat. Nos. 8,522,363, 7,378,043; 7,771,644; 7,814,570; incorporated by reference in entirety.

TABLE 1

| | PI | | PC | | NR | |
|---|---|---|---|---|---|---|
| | (PHR) | | (PHR) | | (PHR) | |
| Component | Min | Max | Min | Max | Min | Max |
| Latex | | | | | | |
| Natural Rubber (NR) | — | — | — | — | 100.000 | — |
| Synthetic Polyisoprene (PI) | 100.000 | — | — | — | — | — |
| Polychloroprene (PC) | — | — | 100.000 | — | — | — |
| Accelerator | | | | | | |
| Zinc Diethyl | 0.300 | 0.700 | — | — | — | — |
| Dithiocarbamate | | | | | | |
| Zinc Dibutyl | 0.060 | 0.130 | — | — | — | — |
| Dithiocarbamate | | | | | | |
| Sodium Dibutyl | 0.700 | 0.120 | — | — | 0.085 | 0.140 |
| Dithiocarbamate | | | | | | |
| Zinc 2- | 0.160 | 0.230 | — | — | — | — |
| mercaptobenzothiazole | | | | | | |
| Dipentamethylene Thiuram | 0.270 | 0.350 | — | — | — | — |
| Polysulphide | | | | | | |
| Diphenyl Guanidine | — | — | — | — | — | — |
| Diisopropyl Xanthogen | 0.270 | 0.350 | — | — | 0.620 | 0.980 |
| Polysulphide | | | | | | |
| N,N′-diphenyl thiourea | 0.170 | 0.250 | — | — | — | — |
| Activator | | | | | | |
| Zinc Oxide Active | 0.620 | 0.690 | 3.250 | 8.400 | 0.380 | 0.790 |
| Vulcanizing agent | | | | | | |
| Sulphur | 0.900 | 1.700 | 0.950 | 1.750 | 0.980 | 1.470 |
| Antioxidant | | | | | | |
| 2,2′-methylene-bis (4-ethyl- | — | — | — | — | — | — |
| 6-tert-butylphenol) | | | | | | |
| 4-and 5-methyl-2- | 0.650 | 1.500 | 0.630 | 1.260 | 0.380 | 0.820 |
| mercaptobenzimidazole | | | | | | |
| Butylated Reaction Product | 1.800 | 2.500 | 0.770 | 1.420 | 0.440 | 0.690 |
| of p-Cresol and | | | | | | |
| Dicyclopentadiene | | | | | | |
| Pigment (optional) | | | | | | |
| Titanium dioxide | 0.250 | 0.750 | 0.210 | 0.560 | 0.164 | 0.235 |
| Copper phthalocyanine | — | — | 0.192 | 0.237 | — | — |
| pigment | | | | | | |
| Surfactant | | | | | | |
| Sodium Linear Alkyl | 0.023 | 0.057 | 0.031 | 0.740 | — | — |
| Benzene Sulfonate | | | | | | |
| Potassium Laurate | — | — | — | — | 0.410 | 0.630 |
| Ethoxylated Alcohol | 0.002 | 0.005 | 0.004 | 0.007 | — | — |
| Anti-wetting agent | | | | | | |
| Polydimethylsiloxane | 0.050 | 0.170 | 0.084 | 0.146 | 0.133 | 0.212 |
| Total Solids Content (TSC) | 30.0 | 35.0 | 37.0 | 45.0 | 33.0 | 42.0 |
| (%) | | | | | | |

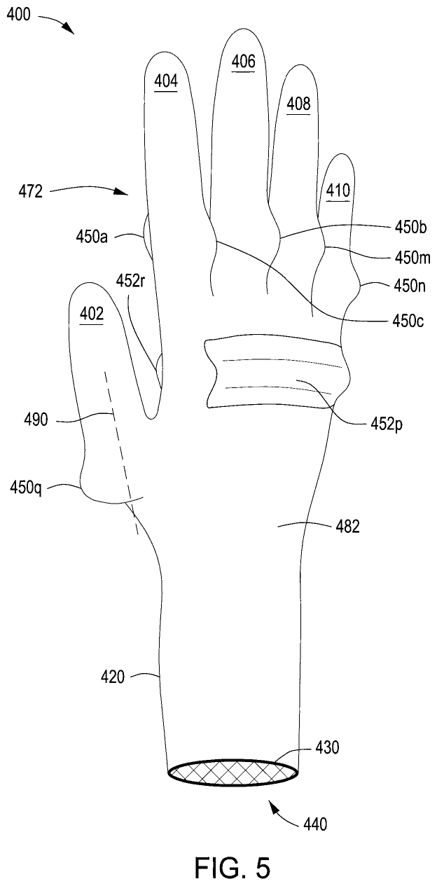

FIG. 5 depicts a perspective view of stress-reducing ridges 450 disposed on a second ambidextrous stress-reducing polymeric glove 400, according to embodiments of the disclosure. As described above, the ambidextrous stress-reducing glove 400 comprises a thumb 402, a plurality of fingers 404, 406, 408, 410, a backhand region 482, a palmside region 472, and a cuff 420. Each of the thumb 402, and the plurality of fingers 404, 406, 408, 410 comprises a backhand surface and a palmside surface. The ambidextrous stress-reducing glove 400 may comprise a bead 430. The ambidextrous stress-reducing polymeric glove 400 defines a volume 440 for receiving a hand of a wearer. Any of the thumb 402 or plurality of fingers 404, 406, 408, 410, the palmside region 472, and/or the backhand region 482, may comprise one or more stress-reducing ridges 450. As shown, there is an elongate stress-reducing ridge 452p under a ridge 450n on the little finger 410, a ridge 450m on the ring finger 408, a ridge 450b on the middle finger 406, and a ridge 450c on the index finger 404. The ambidextrous stress-reducing polymeric glove 400 may comprise an elongate stress-reducing ridge 452r on the palmside region 472. The ambidextrous stress-reducing polymeric glove 400 may comprise an elongate stress-reducing ridge 452p on the backhand side 482. The elongate stress-reducing ridge 452p and the elongate stress-reducing ridge 452r correspond with the metacarpophalangeal joints (MCP).

The elongate stress-reducing ridge 450r and the elongate stress-reducing ridge 452p may be approximately 50 to 80 mm in width and approximately 10 to 30 mm in length. The elongate stress-reducing ridge 452r and the elongate stress-reducing ridge 452p may be approximately 2 to 15 mm in height. The elongate stress-reducing ridge 452r and the elongate stress-reducing ridge 452p may be approximately 12 to 25 mm in distance from crotches formed between the index finger 404, the middle finger 406, the ring finger 408, and the little finger 410. The ridge 450r may be substantially similar to the elongate stress-reducing ridge 452p. The ambidextrous stress-reducing polymeric glove 400 may comprise a ridge 450q on the thumb 402. The ridge 450q may be approximately 15 to 30 mm in width and 15 to 30 mm in length. The ridge 450q may be a bulbous contiguous ridge and traverse approximately 30%-80% of a surface of a circumference of the thumb 402. Some embodiments of the disclosure comprise a ridge 450q that may be a bulbous contiguous ridge and traverse approximately 50% of a surface of a circumference of the thumb 402. As shown, the ridge 450q traverses a midline 490 of the thumb 402 on the backhand side 482 to approximately half the circumference to the palmside 472. It is to be understood that the dimensions described above may be increased, for example, for large and extra-large sized ambidextrous stress-reducing gloves without departing from the scope of the disclosure. It is to be understood that the backhand region 482 may comprise a ridge 450 on the middle finger 406, the ring finger 408 and/or the little finger 410.

At least some of the exemplary embodiments of the disclosure comprises a stress-reducing ridge 450 on all of the backhand surface of the thumb 402 and the palmside surface of the thumb 402, the backhand surface of the index finger 404 and the palmside surface of the index finger 404, the backhand surface of the middle finger 406 and the palmside surface of the middle finger 406, the backhand surface of the ring finger 408 and the palmside surface of the ring finger 408, the backhand surface of the little finger 410 and the palmside surface of the little finger 410, and the palm region 472 and the backhand region 482. At least some of the exemplary embodiments of the disclosure comprises a stress-reducing ridge 450 on all of the backhand surface of the thumb 402 and the palmside surface of the thumb 402, the backhand surface of the index finger 404 and the palmside surface of the index finger 404, the backhand surface of the middle finger 406 and the palmside surface of the middle finger 406, the backhand surface of the ring finger 408 and the palmside surface of the ring finger 408, the backhand surface of the little finger 410 and the palmside surface of the little finger 410, and the palm region 472 and the backhand region 482 and an elongate stress-reducing ridge 452r on the palmside region 472 and an elongate stress-reducing ridge 452p on the backhand region 482.

Ambidextrous stress-reducing gloves, such as the ambidextrous stress-reducing polymeric glove 100, 400 that comprise a ridge, e.g., the stress-reducing ridge 150, 450 and/or 452 have a curved length across the ridge as described further below with respect to glove formers used to make the ambidextrous stress-reducing polymeric glove 100, 400. The ambidextrous stress-reducing polymeric glove 100, 400 can flex easily at the distal interphalangeal joint, proximal interphalangeal joint, and/or metacarpophalangeal joint of a hand of a wearer wearing the ambidextrous stress-reducing polymeric glove 100, 400 without having to significantly stretch the elastomeric material of which the ambidextrous stress-reducing polymeric glove 100, 400 is made. The ambidextrous stress-reducing glove may be made from any of the thermoplastics or thermoplastic elastomers described above. Exemplary elastomers for making the ambidextrous stress-reducing polymeric glove 400 having stress-reducing ridges 450, 452 are described in Table 1.

The ridges 150, 450, 452 as with all other stress-reducing ridges described herein, provide additional glove material at the joints so that a person wearing the ambidextrous stress-reducing polymeric glove 200 may open and close the hand, i.e., flex, without significantly stretching the material in areas of the ambidextrous stress-reducing glove 200 corresponding to the joints. It should be noted that the foregoing dimensions are for a size 7.5 ambidextrous stress-reducing glove. The stress-reducing ridges 150, 450, 452 and all other stress-reducing ridges described herein may be made larger or smaller, as appropriate for an application.

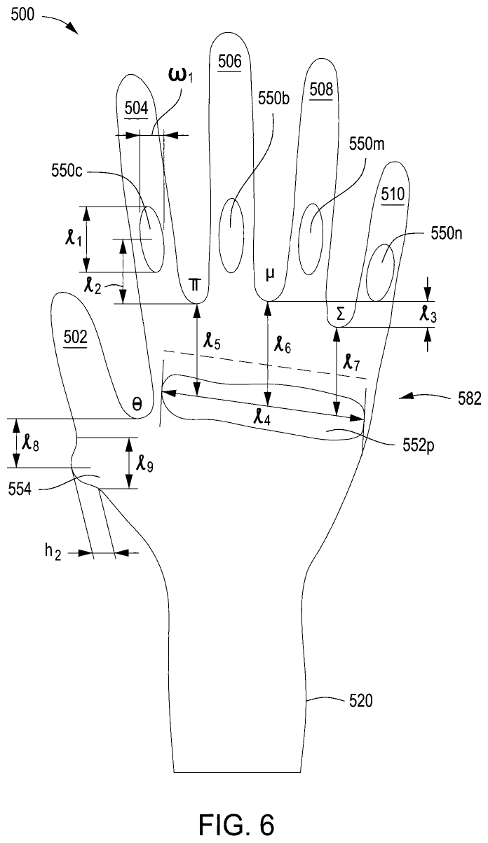

FIG. 6 depicts a front plan view of a glove former 500 for manufacturing an ambidextrous stress-reducing polymeric glove, such as the ambidextrous stress-reducing polymeric glove 400 having stress-reducing ridges 450, 452 described above, according to embodiments of the disclosure. The glove former 500 comprises a thumb region 502, a plurality of finger regions 504, 506, 508, 510, a palmside region (not shown), a backhand region 582, and a cuff region 520. The glove former 500 is a negative of the stress-reducing polymeric glove 400 as described above. Any of the thumb region 502 or the plurality of fingers 504, 506, 508, 510, the palmside region, and/or the backhand region 582, may comprise one or more former ridges 550, 552, 554. As shown, there is an elongate former ridge 552p under the little finger 510, the ring finger 508, the middle finger 506, and the index finger 504. The glove former 500 may comprise an elongate former ridge on the palmside region (not shown).

As depicted, the plurality of fingers 504, 506, 508, 510 comprise a former ridge 550c, 550b, 550m, and 550n, respectively. The thumb region 502 comprises a former ridge 554. The former ridge 554 may comprise a shape similar to former ridge 550c, 550b, 550m, and 550n. Alternatively, the former ridge 554 may comprise a truncated toroid that traverses 30-70% of the circumference of the thumb 502. Some exemplary embodiments of the disclosure comprise a former ridge 554 traversing 50% of a circumference of the thumb 502. The truncated toroid can be defined as a bulbous former ridge, such as the former ridge 554. A bulbous former ridge, i.e., no sharp inflections, facilitates former cleaning. A glove former 500 that is clean eliminates or substantially eliminates quality defects due to a polymeric composition, such as in Table 1, sticking to the glove former(s). It is to be understood that corresponding former ridges 550, 552, 554 are present on the palmside region on each of the plurality of fingers 504, 506, 508, 510, the thumb 502, and the MCP joint area of the palmside region.

The former ridge 554 comprises a ridge height h₂ of approximately 2-15 mm. The former ridge 554 comprises a ridge length l₉ of approximately 15-30 mm. The former ridge 554 is approximately a length l₈ of approximately 10-25 mm from the thumb crotch Θ. The former ridges 550c, 550b, 550m, and 550n comprise a height of 1-20 mm. The former ridges 550c, 550b, 550m, and 550n comprise a length l₁ of approximately 10-30 mm and a width ω₁ of approximately 5-25 mm. The former 500 also comprises a first crotch π, a second crotch μ, a third crotch Σ, and a thumb crotch Θ. The former ridge 550c has a peak, approximately at the midpoint of the former ridge 550c, that is located a length l₂ of approximately 10-25 mm from an axis formed by a line through the first crotch π. The former ridge 550b has a peak, approximately at the midpoint of the former ridge 550b, that is located a length of approximately 10-25 mm from an axis formed by a line through the second crotch μ. The ridge 550m has a peak, approximately at the midpoint of the former ridge 550m, that is located a length of approximately 10-25 mm from an axis formed by a line through the a second crotch μ. The former ridge 550n has a peak, approximately at the midpoint of the former ridge 550n, that is located a length l₃ of approximately 5-15 mm from an axis formed by a line through the third crotch Σ.

The former ridge 552p comprises a height of approximately 2-15 mm. The former ridge 552p comprises a length l₄ of approximately 50-80 mm. The former ridge 552p is a length l₅ of approximately 15-25 mm from the first crotch m, a length l₆ of approximately 15-25 mm from a second crotch μ, a length l₇ of approximately 12-25 mm a third crotch Σ. It is to be understood that the foregoing dimensions are for a size 7.5 ambidextrous stress-reducing glove. The former ridges 550, 552, 554 may be made larger or smaller. Furthermore, the glove itself may be larger or smaller and the dimensions and sizes of the former ridges 550, 552, 554 and/or lengths therebetween l₁-l₉ may be larger or smaller as appropriate for larger or smaller gloves.

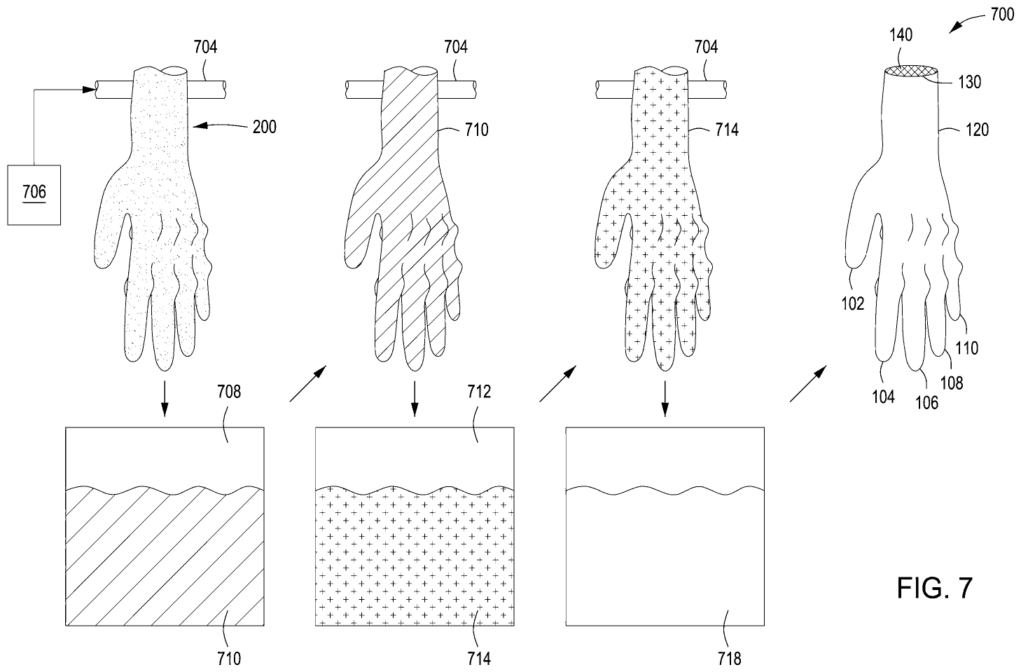

FIG. 7 depicts an apparatus 700 for forming an ambidextrous stress-reducing polymeric glove having stress-reducing ridges, according to embodiments of the disclosure. A glove former 200, substantially as described above (or the glove former 500), i.e., having finger regions a thumb region, and a plurality of features 150, 450 for forming stress-reducing ridges is provided along a conveyor 704, further comprising a controller 706 for controlling the movement of the former(s) 200, 500 and the conveyor 504. The glove former 200, 500 has a ridge forming features 250, 550, 552, 554 and other former ridges, as described above, that are configured to allow excess coagulant solution or polymeric emulsion to drip off in a manner that avoids uneven thickness of both the coagulant solution and/or polymeric emulsion.

The glove former 200, 500 is dipped into a tank 708 containing a coagulant solution 710. The glove former 200, 500 is removed and allowed to drip dry, leaving coagulant solution 710 on the glove former 200, 500. The conveyor 704 may allow the former 200, 500 to drip dry with the fingers pointing downward and/or rotate so that the fingers are pointed upward. The glove former 200, 500 is next dipped into a tank 712 having an elastomeric emulsion 714, e.g., a polymeric composition, as described above in Table 1, therein. The glove former 200, 500 is removed and the elastomeric emulsion 714 allowed to drip from the glove former 200, 500, leaving an elastomeric coating 716 on the glove former 200, 500, which again may optionally be pointed downward followed by rotation so that the fingers are pointed upward. The former 200, 500 having the elastomeric coating 716 is delivered to an oven 718 so that the elastomeric coating 716 on the glove former 200, 500 is heated and cured, forming an ambidextrous stress-reducing polymeric glove 100 (or 400), which comprises a shape that is the negative of the glove former 200, 500. The ambidextrous stress-reducing polymeric glove 100, 400 is then stripped from the former 200, 500. As shown, the ambidextrous stress-reducing polymeric glove 100 comprises a cuff 130, a thumb 102, a plurality of fingers 104, 106, 108 and 110, a backhand side region and a bead 130. As shown, the ambidextrous stress-reducing polymeric glove 100 comprises ridges 150 on each of the plurality of fingers 104, 106, 108, and 110, and the backhand side region 182.

Glove formers described herein, such as the glove former 200, 500, in accordance with embodiments of the disclosure may be made of ceramics, borosilicates, metals, plastics, and other suitable materials. Elastomers in accordance with embodiments of the disclosure comprise natural rubber latex, synthetic polyisoprene, polychloroprene, butyl rubbers, nitrile-butadienes, highly carboxylated nitrile-butadienes (such as nitrile-butadienes having 35% or greater carboxylation), copolymers of styrene-butadienes, and blends or mixtures thereof.

Embodiments according to the disclosure comprise the use of a coagulant solution to wet the former and may include an exemplary aqueous solution of 5% calcium nitrate, although other concentrations are possible as are known to those in the art, such as an aqueous solution of 3.5-15% calcium nitrate. Other salts, such as calcium chloride, calcium citrate, aluminum sulfate, and the like and/or mixtures thereof may be used. Furthermore, the coagulant solution may be aqueous, alcoholic, or a mixture of aqueous and alcoholic solutions/solvents. Weaker acid solutions may also be used as coagulants, such as formic acid, acetic acid, and other low pKa acids as are known to those in the art. Exemplary embodiments include the disposition of a weak acid solution onto a former, coating the former with a polymeric emulsion, treating the coating with a weak acid solution followed by treating the coating with a strong acid/coagulant solution.

Although some embodiments have been discussed above, other implementations and applications are also within the scope of the following claims. The disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the following claims. Also, as used in this specification and the appended claims, the singular forms “a,” “an,” and “the” include plural referents unless the context clearly dictates otherwise. Reference throughout this specification to “some embodiments,” “at least one embodiment,” “one embodiment” or “an embodiment” means that a particular feature, structure, material, step, or characteristic described in connection with the embodiment is included in at least one embodiment of the disclosure, but do not denote that they are necessarily present in every embodiment. Therefore, the recitation of the phrases “some embodiments,” “at least one embodiment,” “in one embodiment” or “in an embodiment” throughout this specification are not necessarily referring to the same embodiment of the disclosure. Nonetheless, it is to be understood that any and all features, structures, materials, steps, and/or characteristics may be included in any embodiment except where specifically excluded.

“Optional” or “optionally” means that the subsequently described event or feature may or may not occur or be necessary for any embodiment, or that the subsequently identified material may or may not be present, and that the description includes instances where the event or feature occurs or where the material is present, and instances where the event or feature does not occur or the material is not present.

As used herein, the terms “comprises,” “comprising,” “includes,” “including,” “has,” “having,” or any other tense thereof, are intended to cover a non-exclusive inclusion. For example, a process, method, article or apparatus that comprises a list of elements is not necessarily limited to only those elements, but may include other elements not expressly listed or inherent to such process, method, article, or apparatus.

As used herein, throughout the specification and claims, approximating language may be applied to modify any quantitative representation that could permissibly vary without resulting in a change in the basic function to which it is related. Accordingly, a value modified by a term or terms, such as “about” or “approximately,” is not limited to the precise value specified. In at least some instances, the approximating language may correspond to the precision of an instrument for measuring the value. Range limitations may be combined and/or interchanged, and such ranges are identified and include all the sub-ranges stated herein unless context or language indicates otherwise. Other than in the operating examples or where otherwise indicated, all numbers or expressions referring to quantities of ingredients, reaction conditions and the like, used in the specification and the claims, are to be understood as modified in all instances by the term “about” or “approximately.”

All ranges recited herein include ranges therebetween, and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more.

To facilitate understanding, identical reference numerals have been used, where possible, to designate comparable elements that are common to the figures. The figures are not drawn to scale and may be simplified for clarity. It is contemplated that elements and features of one embodiment may be beneficially incorporated in other embodiments without further recitation.

It is to be understood that various changes and modifications to the embodiments described herein will be apparent to those skilled in the art. Such changes and modifications can be made without departing from the spirit and scope of the present disclosure and without demising the attendant advantages. It is, therefore, intended that such changes and modifications be covered by the appended claims.

All references, including publications, patent applications, and patents, cited herein are incorporated by reference to the same extent as if each reference were individually and specifically indicated to be incorporated by reference and were set forth in its entirety herein.

1. An ambidextrous stress-reducing polymeric glove, comprising:
a thumb having a palmside surface and a backhand surface;
a plurality of fingers that includes an index finger, a middle finger, a ring finger, and a little finger, each of the plurality of fingers having a palmside surface and a backhand surface;
a palm region;
a backhand region; and
a stress-reducing ridge on at least one of:
the backhand surface of the thumb and the palmside surface of the thumb,
the backhand surface of the index finger and the palmside surface of the index finger,
the backhand surface of the middle finger and the palmside surface of the middle finger,
the backhand surface of the ring finger and the palmside surface of the ring finger,
the backhand surface of the little finger and the palmside surface of the little finger, or
the palm region and the backhand region.

2. The ambidextrous stress-reducing polymeric glove of claim 1, wherein the stress-reducing ridge on the palm region and the backhand region is an elongate stress-reducing ridge.

3. The ambidextrous stress-reducing polymeric glove of claim 1, wherein a position of the stress-reducing ridge corresponds to at least one of a distal interphalangeal joint, a proximal interphalangeal joint, and/or a metacarpophalangeal joint of a human hand.

4. The ambidextrous stress-reducing polymeric glove of claim 1, wherein a bulbous contiguous stress-reducing ridge traverses at least 50% of a circumference of the thumb.

5. The ambidextrous stress-reducing polymeric glove of claim 2, wherein the elongate stress-reducing ridge is 2-15 mm in height, 10-30 mm in width, and 50-80 mm in length.

6. The ambidextrous stress-reducing polymeric glove of claim 2, wherein the elongate stress-reducing ridge is 12-25 mm in distance from a finger crotch.

7. The ambidextrous stress-reducing polymeric glove of claim 1, wherein the stress-reducing ridge is 1-20 mm in height.

8. The ambidextrous stress-reducing polymeric glove of claim 1, wherein the stress-reducing ridge is 10-30 mm in length.

9. The ambidextrous stress-reducing polymeric glove of claim 1, wherein the stress-reducing ridge is 5-25 mm in width.

10. The ambidextrous stress-reducing polymeric glove of claim 1, wherein a stress-reducing ridge is on all of the backhand surface of the thumb and the palmside surface of the thumb, the backhand surface of the index finger and the palmside surface of the index finger, the backhand surface of the middle finger and the palmside surface of the middle finger, the backhand surface of the ring finger and the palmside surface of the ring finger, the backhand surface of the little finger and the palmside surface of the little finger, and the palm region and the backhand region.

11. The ambidextrous stress-reducing polymeric glove of claim 10, wherein the stress-reducing ridge is 1-20 mm in height.

12. A glove former for manufacturing a polymeric glove, comprising:
a thumb having a palmside surface and a backhand surface;
a plurality of fingers that includes an index finger, a middle finger, a ring finger, and a little finger, each of the plurality of fingers having a palmside surface and a backhand surface;
a palm region;
a backhand region; and
a stress-reducing former ridge on at least one of:
the backhand surface of the thumb and the palmside surface of the thumb,
the backhand surface of the index finger and the palmside surface of the index finger,
the backhand surface of the middle finger and the palmside surface of the middle finger,
the backhand surface of the ring finger and the palmside surface of the ring finger,
the backhand surface of the little finger and the palmside surface of the little finger, or
the palm region and the backhand region, wherein the stress-reducing former ridges are 1-20 mm in height, and a placement of the stress-reducing former ridges correspond to at least one of a distal interphalangeal joint, a proximal interphalangeal joint, and/or a metacarpophalangeal joint of a hand.

13. The glove former for manufacturing a polymeric glove of claim 12, wherein a stress-reducing former ridge is on all of the backhand surface of the thumb and the palmside surface of the thumb, the backhand surface of the index finger and the palmside surface of the index finger, the backhand surface of the middle finger and the palmside surface of the middle finger, the backhand surface of the ring finger and the palmside surface of the ring finger, the backhand surface of the little finger and the palmside surface of the little finger, and the palm region and the backhand region.

14. The glove former for manufacturing a polymeric glove of claim 12, comprising an elongate former ridge on a palmside region and a backhand region.

15. The glove former for manufacturing a polymeric glove of claim 12, wherein a bulbous contiguous stress-reducing former ridge traverses at least 50% of the palmside surface of the thumb.

16. A method for manufacturing a stress-reducing polymeric glove, comprising:
dipping a hand-shaped former into a polymeric emulsion to form a polymeric coating disposed upon the hand-shaped former, wherein the hand-shaped former comprises a thumb having a palmside surface and a backhand surface; a plurality of fingers that includes an index finger, a middle finger, a ring finger, and a little finger, each of the plurality of fingers having a palmside surface and a backhand surface; a palm region; a backhand region; and a stress-reducing former ridge on at least one of: the backhand surface of the thumb and the palmside surface of the thumb, the backhand surface of the index finger and the palmside surface of the index finger, the backhand surface of the middle finger and the palmside surface of the middle finger, the backhand surface of the ring finger and the palmside surface of the ring finger, the backhand surface of the little finger and the palmside surface of the little finger, or the palm region and the backhand region, wherein the stress-reducing former ridges are 1-20 mm in height, and a placement of the stress-reducing former ridges correspond to at least one of a distal interphalangeal joint, a proximal interphalangeal joint, and/or a metacarpophalangeal joint of a hand; and
curing the polymeric coating to form a stress-reducing polymeric glove.

17. The method of claim 16, wherein the hand-shaped former comprises a stress-reducing former ridge is on all of the backhand surface of the thumb and the palmside surface of the thumb, the backhand surface of the index finger and the palmside surface of the index finger, the backhand surface of the middle finger and the palmside surface of the middle finger, the backhand surface of the ring finger and the palmside surface of the ring finger, the backhand surface of the little finger and the palmside surface of the little finger, and the palm region and the backhand region.

18. The method of claim 16, wherein the hand-shaped former comprises an elongate former ridge on a palmside region and a backhand region.

19. The method of claim 16, wherein the hand-shaped former comprises a bulbous contiguous stress-reducing former ridge traverses at least 50% of the palmside surface of the thumb.

20. The method of claim 16, wherein curing comprises heating the coating to a temperature of approximately 110-130 degrees Celsius.
